(19) United States
(12) Patent Application Publication
(10) Pub. No.: US 2020/0300773 A1
(43) Pub. Date: Sep. 24, 2020

(54) Colorimetric sensor chip for gas sensing

(71) Applicant: TAIWAN CARBON NANO TECHNOLOGY CORPORATION, Miaoli County (TW)

(72) Inventors: Ching-Tung HSU, MIAOLI COUNTY (TW); Chun-Wei SHIH, MIAOLI COUNTY (TW); Kuang-Che LEE, MIAOLI COUNTY (TW); Chia-Hung LI, MIAOLI COUNTY (TW); Chien-Yao HUANG, MIAOLI COUNTY (TW); Chun-Hsien TSAI, MIAOLI COUNTY (TW); Ting-Chuan LEE, MIAOLI COUNTY (TW); Chun-Jung TSAI, MIAOLI COUNTY (TW)

(21) Appl. No.: 16/825,060

(22) Filed: Mar. 20, 2020

(30) Foreign Application Priority Data
Mar. 22, 2019 (TW) 108110102

(51) Int. Cl. G01N 21/78; G01N 33/52

(52) U.S. Cl. CPC G01N 21/783; G01N 33/525

(57) ABSTRACT

The present invention relates to a colorimetric sensor chip includes a chemical reaction layer and a coloring reaction layer. The chemical reaction layer includes reaction zones reacting with a gas to be tested to produce a chemical change. The coloring reaction layer includes a coloring side and a reaction side in contact with the reaction zone which are opposite to each other. The coloring reaction layer further includes a coloring indicator to produce a coloring reaction corresponding to the chemical change of the reaction sides, thereby completing a light, thin and highly integrated gas sensor chip directly attaching or placing on an object to be sensed for real-time sensing.

### FIELD OF THE INVENTION

The present invention relates to a sensor chip, and more particularly to a light, thin and highly integrated colorimetric sensor chip.

### BACKGROUND OF THE INVENTION

In recent years, gas sensing devices used to detect the flow rate and type of gas become thinner and lighter. The dimensions of the gas sensing devices have been greatly reduced to less than 1 cm in the form of chip, and the integration with other devices has also been greatly improved. However, such a type of gas sensing chip integrated with other devices has a complicated structure, and usually includes a plurality of sensor arrays internally. Although the electric current transmission of each of the sensors in the array is controlled independently according to the current semiconductor technology, and the problem of the bus is solved, the drawbacks of high temperature and large power consumption still need to be overcome.

Another type of gas sensing device has a relatively simple structure. For example, the Taiwan patent no. 1374265 mentions a gas sensor, which includes a planar inductance-capacitance resonator and a gas absorbing material. The planar inductance-capacitance resonator includes an inductance electrode and a capacitance electrode, and the capacitance electrode is connected to the inductance electrode. The gas absorbing material is connected to at least a part of the capacitance electrode. Through the above structure, the gas absorbing material changes the resonance frequency of the planar inductance-capacitance resonator according to a change in the concentration of a gas to be tested, and then the change in the concentration of the gas to be tested is known.

However, such a type of gas sensing device still needs to rely on power supply, so the applicable range is relatively limited.

### SUMMARY OF THE INVENTION

An object of the present invention is to solve the drawbacks of high temperature and large power consumption of the conventional energized gas sensing chips during operation, and the application field is restricted since the conventional energized gas sensing chips must require power supply during the measurement. Another object of the present invention is to provide a light, thin and highly integrated gas sensing chip.

In order to achieve the above objects, the present invention provides a colorimetric sensor chip including a chemical reaction layer and a coloring reaction layer. The chemical reaction layer includes at least one reaction zone reacting with a gas to be tested to produce a chemical change, and one side of the chemical reaction layer opposite to the coloring reaction layer is an air inlet side. The coloring reaction layer includes a coloring side and a reaction side opposite to each other, the reaction side contacts with the reaction zone of the chemical reaction layer. The coloring reaction layer includes a coloring indicator to produce a coloring reaction corresponding to the chemical change of the reaction side.

Accordingly, the colorimetric sensor chip of the present invention reacts with the gas to be tested through the reaction zones disposed on the chemical reaction layer, and then undergoing the chemical change. The chemical change shows different colors through the reaction of the coloring indicator of the coloring reaction layer. Users judge the colors with an existing database or through digitization. In this way, the colorimetric sensor chip of the present invention completes gas sensing without consuming electric power. Besides, the colorimetric sensor chip performs real-time sensing by directly attaching or placing on an object to be sensed due to its simple, light and thin structure.

### BRIEF DESCRIPTION OF THE DRAWINGS

FIG. 1 is a schematic diagram of a first embodiment of a colorimetric sensor chip of the present invention;

FIG. 2 is a schematic diagram of a second embodiment of the colorimetric sensor chip of the present invention;

FIG. 3 is a schematic diagram of a third embodiment of the colorimetric sensor chip of the present invention;

FIG. 4 is a schematic diagram of a fourth embodiment of the colorimetric sensor chip of the present invention;

FIG. 5 is a schematic diagram of a fifth embodiment of the colorimetric sensor chip of the present invention;

FIG. 6 is a schematic diagram of a sixth embodiment of the colorimetric sensor chip of the present invention;

FIG. 7 is a schematic diagram of a coloring side of the colorimetric sensor chip of the present invention;

FIG. 8 is a schematic diagram of a method for manufacturing the colorimetric sensor chip according to an embodiment of the present invention; and

FIG. 9 is a schematic diagram of a method for manufacturing the colorimetric sensor chip according to another embodiment of the present invention.

### DETAILED DESCRIPTION OF THE PREFERRED EMBODIMENTS

The detailed description and technical contents of the present invention are described below with reference to the drawings.

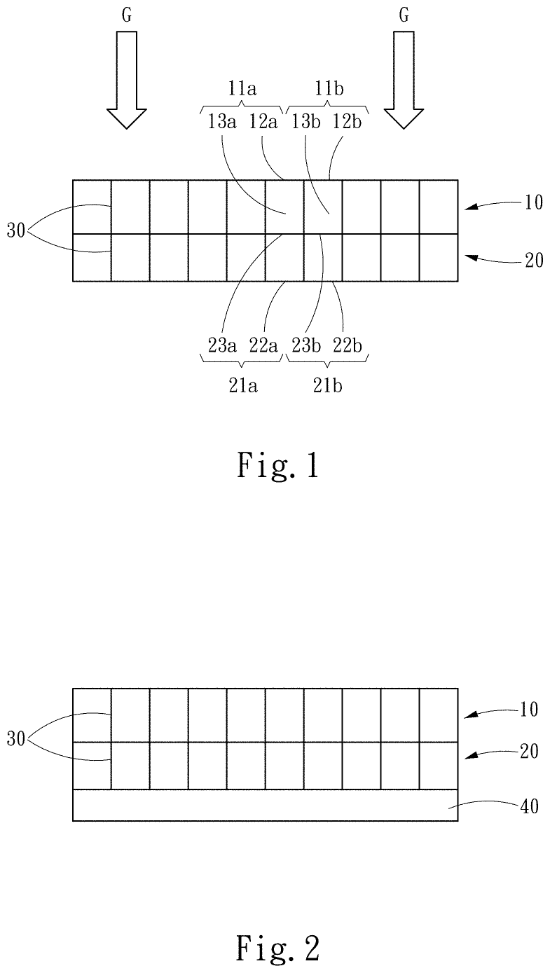

FIG. 1 is a schematic diagram of a first embodiment of a colorimetric sensor chip. The colorimetric sensor chip mainly includes a chemical reaction layer 10, a coloring reaction layer 20 stacked with the chemical reaction layer 10, and a plurality of partition portions 30.

In this embodiment, the chemical reaction layer 10 is divided into a plurality of first areas by the partition portions 30, wherein the first areas 11a, 11b marked in FIG. 1 are only used as an example for illustration in the embodiment. The first areas 11a, 11b respectively include air inlet sides 12a, 12b disposing on sides opposite to coloring reaction layer 20, and reaction zones 13a, 13b. A gas to be tested G enters into the reaction zones 13a, 13b through the air inlet sides 12a, 12b, and the reaction zones 13a, 13b react with the gas to be tested G to produce a chemical change. The reaction zones 13a, 13b respectively includes different kinds of chemicals to react with the different gases to be tested G. For example, some of the reaction zones 13a, 13b react with alkanes, some of the reaction zones 13a, 13b react with alcohols, and some of the reaction zones 13a, 13b react with sulfides. The partition portions 30 separate the adjacent first areas 11a, 11b so that reactions occurring in the adjacent first areas 11a, 11b do not affect each other. The chemical change is produced by a redox reaction, an acid-base reaction, an enzyme-catalytic reaction, a metal-catalytic reaction, a condensation reaction, a hydrolysis reaction, an addition reaction, an elimination reaction, a substitution reaction, or combinations of the above, but is not limited thereto. For example, one suitable redox reaction for the present invention could be the oxidizing ethanol to acetaldehyde or acetic acid, and a glucose oxidase is used in enzyme-catalytic reaction, and a platinum catalyst is used in metal catalyst.

In this way, assuming that one of the reaction zones 13a, 13b is coated with hydrazine (H₂N—NH₂), carbazic acid (H₂NNHCOOH) is produced when the gas to be tested G containing carbon dioxides reacts with the reaction zones 13a, 13b coated with hydrazine, and color is generated by using crystal violet as a redox indicator. Also, in an embodiment, the colorimetric sensor chip further includes a protective layer (not shown in the figure) disposed on the air inlet sides 12a, 12b to prevent gas from directly entering the reaction zones 13a, 13b to cause interference or damage.

The coloring reaction layer 20 is also divided into a plurality of second areas by the partition portions 30, wherein the second areas 21a, 21b marked in FIG. 1 are only used as an example for illustration in the embodiment. The second areas 21a, 21b and the first areas 11a, 11b are stacked with each other, and the second areas 21a, 21b include coloring sides 22a, 22b respectively, and reaction sides 23a, 23b respectively contact with the reaction zones 13a, 13b of the chemical reaction layer 10. The coloring reaction layer 20 includes a coloring indicator; therefore, when the chemical change is produced in the reaction zones 13a, 13b due to chemical reactions, the coloring reaction layer 20 in contact with the reaction zones 13a, 13b produces a coloring reaction corresponding to the chemical change.

In this embodiment, the partition portion 30 is a partition wall that separates the adjacent first areas 11a, 11b and the second areas 21a, 21b, so that the reaction zone 13a will not affect the adjacent reaction zone 13b when the gas to be tested G enters through the air inlet side 12a to react with the reaction zone 13a. Likewise, reactions occurred in the reaction zone 13a will only affect the reaction side 23a and the coloring side 22a, but will not affect the reaction side 23b and the coloring side 22b. In addition, in this embodiment, the chemical reaction layer 10 and the coloring reaction layer 20 are a double-layer structure independent of each other. However, in other embodiments, the chemical reaction layer 10 and the coloring reaction layer 20 are a single-layer structure, that is, the chemical reaction layer 10 and the coloring reaction layer 20 are integrated into a single layer.

Compositions of the coloring indicator are selected from a group consisting of a hydrate, a precipitate, a metal complex, and combinations thereof. Take the hydrate as an example, it can be pink hydrate produced when dry cobaltous chloride meets water vapor; take the precipitate as an example, it can be black lead sulfide precipitate produced when lead acetate meets hydrogen sulfide; take the metal complex as an example, it can be oxygen coordinating and combining with iron ions in heme to present bright red color. The “coloring indicator” suitable for use in the present invention is not particularly limited. For example, the coloring indicator is further an acid-base indicator, a solvatochromism, or combinations thereof. For instance, the acid-base indicator suitable for use in the present invention is not particularly limited. In an embodiment, the acid-base indicator is a coloring reagent such as Bromothymol Blue, or phenolphthalein, and the like.

Further, please refer to FIG. 2 for a schematic diagram of a second embodiment of the colorimetric sensor chip. Compared with the first embodiment described above, the second embodiment further includes an anti-reflection film 40 disposed on the coloring sides 22a, 22b. The anti-reflection film 40 helps users to observe changes in color of the coloring sides 22a, 22b from outside through an instrument or the naked eye without interference.

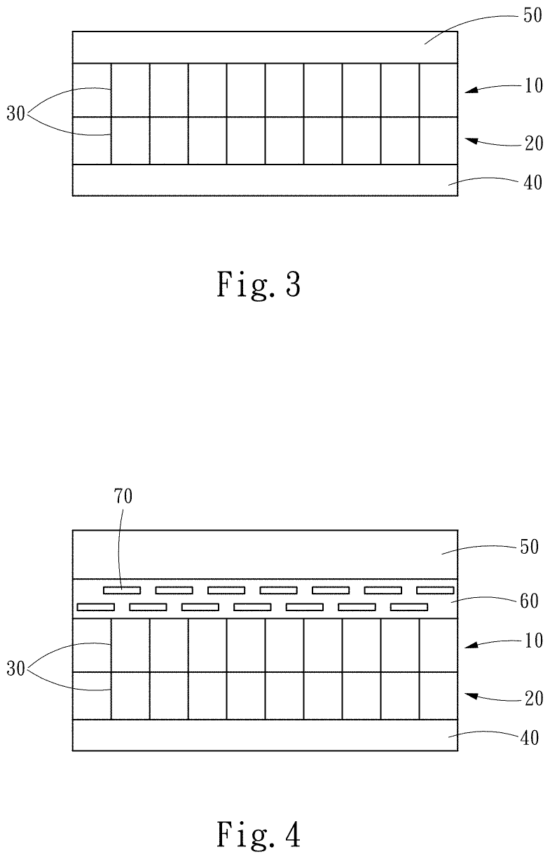

Further, please refer to FIG. 3, in a third embodiment of the colorimetric sensor chip, an air-permeable film 50 with water-blocking property is disposed to reduce the interference of the external environment to the internal chemical reactions, and the air-permeable film 50 is disposed on the air inlet sides 12a, 12b of the chemical reaction layer 10. In FIG. 3, the air-permeable film 50 is provided based on the structure of the second embodiment shown in FIG. 2. In another embodiment, the air-permeable film 50 is provided based on the structure of the first embodiment without limitation.

In FIG. 4, a diffusion film 60 is provided based on the structure of the third embodiment shown in FIG. 3. In a fourth embodiment shown in FIG. 4, at least one layer of diffusion film 60 with gas screening function is sandwiched between the air-permeable film 50 and the chemical reaction layer 10 to achieve the effect of screening specific gases. Moreover, gases targeted by each of the diffusion films 60 are different from each other when the diffusion films 60 are provided. In addition, each of the diffusion films 60 is added with graphenes 70 to adjust the diffusion path of gases in the diffusion films 60, thereby changing the diffusion speeds of large and small molecules to obtain the effect of screening large and small molecules.

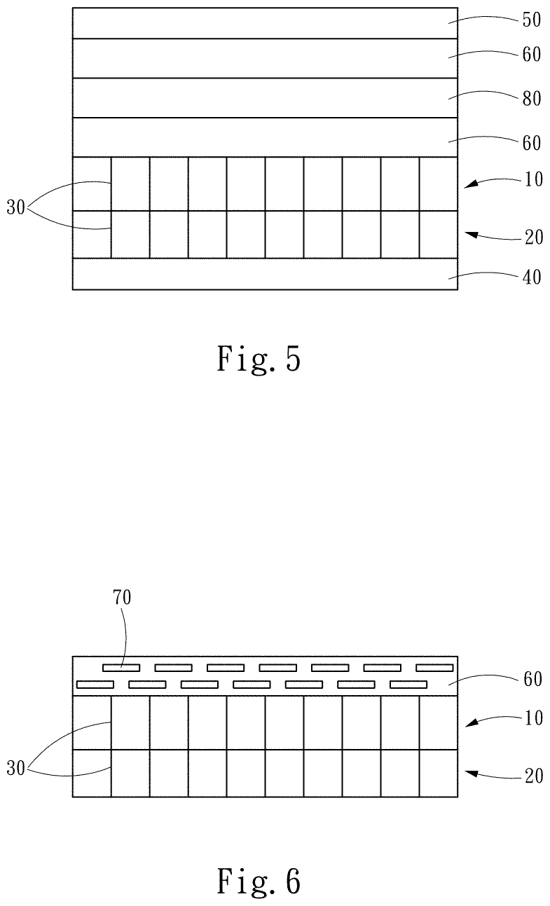

In addition, the colorimetric sensor chip further includes an adsorption molecule (not shown in the figures) in the diffusion film 60 to adsorb gas molecules more efficiently. The adsorption molecule is selected from any liquid, colloid, hole, or fiber film with adsorption function. In an embodiment, glycerin is used as the adsorption molecule; or in an embodiment, holes are used as the adsorption molecule to screen out larger-sized gas molecules by its characteristics. However, in another embodiment, as shown in FIG. 5, an adsorption layer 80 containing adsorption molecules is directly disposed between a pair of the diffusion films 60, thereby having good adsorption effect.

Further, please refer to FIG. 6 for a schematic diagram of a sixth embodiment of the colorimetric sensor chip, the structure of the sixth embodiment is provided based on the structure of the first embodiment. At least one diffusion film 60 with gas screening function is directly formed on the air inlet sides 12a, 12b of the chemical reaction layer 10, and the diffusion film 60 is selectively provided with the graphenes 70 to adjust the diffusion path of gases in the diffusion films 60. The materials and functions of the film layers in this embodiment are the same as the embodiments described above, and will not be described in detail.

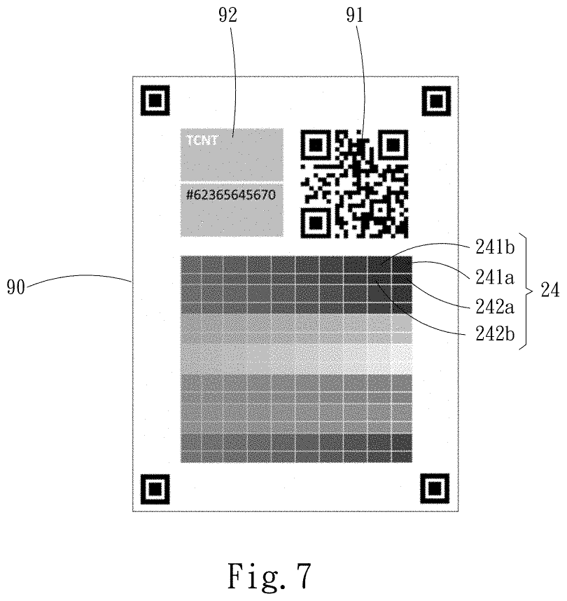

Further, please refer to FIG. 7, in this embodiment, the colorimetric sensor chip shown in FIG. 1 is fixed on a carrier 90, wherein the carrier 90 is a sticker, and a plurality of colorimetric blocks 24 corresponding to the first areas 11a, 11b and the second areas 21a, 21b are formed on the carrier 90. In this embodiment, the colorimetric block 24 includes a plurality of first colorimetric blocks 241a, 241b and a plurality of second colorimetric blocks 242a, 242b. The first colorimetric blocks 241a, 241b and the second colorimetric blocks 242a, 242b have different colors, such as red and yellow, and the first colorimetric blocks 241a, 241b are red with different color ramps respectively, the second colorimetric blocks 242a, 242b are yellow with different color ramps respectively. The colorimetric blocks 24 shown in FIG. 7 are merely illustrative, and are not intended to limit the present invention. Further, in this embodiment, the carrier 90 is further provided with a two-dimensional QR image code 91 and a label 92.

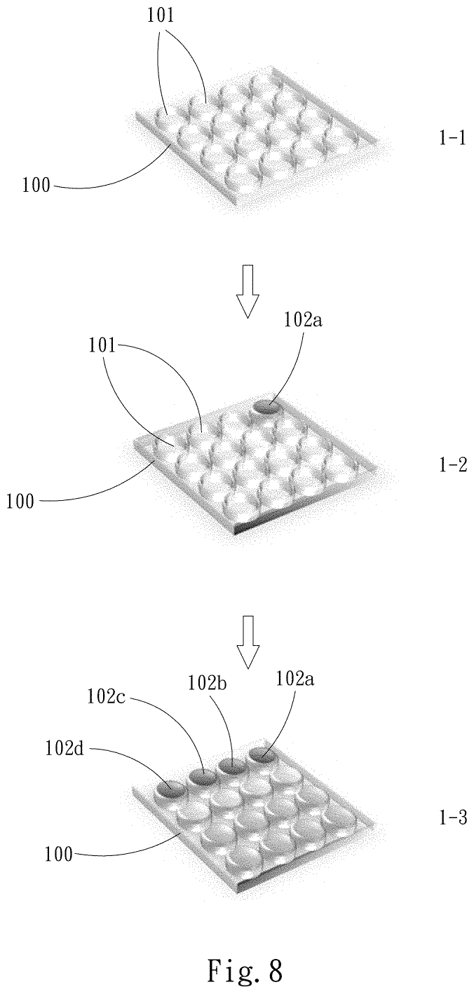

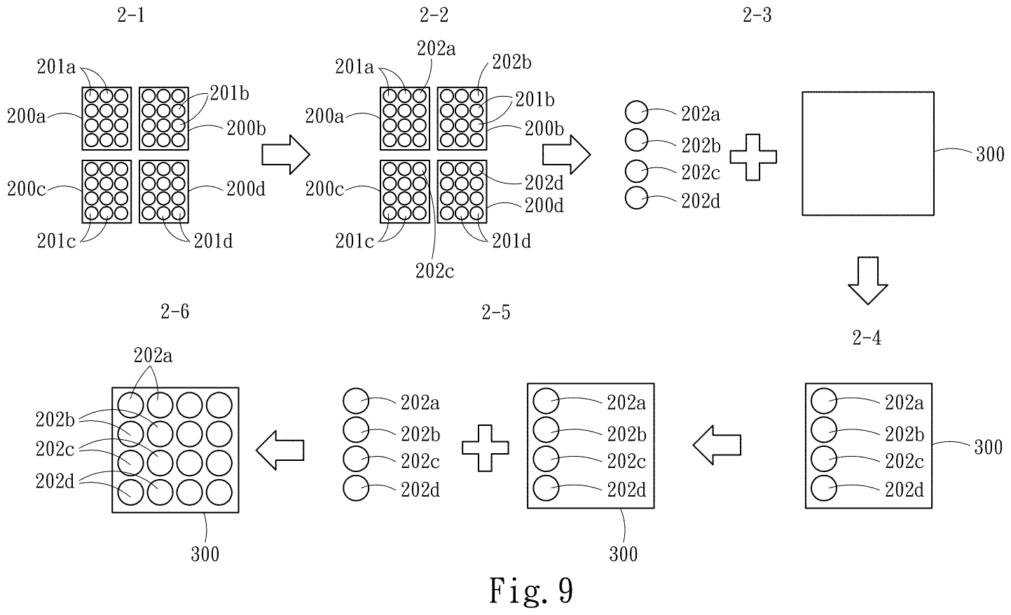

FIG. 8 and FIG. 9 are respectively schematic diagrams of methods for manufacturing the colorimetric sensor chip of the present invention, wherein FIG. 8 is a “bottom up” method, and

FIG. 9 is a “top down” method.

The method shown in FIG. 8 firstly uses a test paper 100 as a substrate (step 1-1), and pretreatment is performed on one side of the test paper 100 to separate into a plurality of blocks 101 that do not affect each other. Subsequently, the coloring reaction layer 20 and the chemical reaction layer 10 are titrated sequentially on one of the blocks 101 and are dried to form a sensing portion 102a (step 1-2). The sensing portion 102a includes the coloring reaction layer 20 and the chemical reaction layer 10 mentioned above. Then, sensing portions 102b, 102c, and 102d with different compositions are formed on the adjacent blocks 101 respectively (step 1-3). The manufacturing method of the colorimetric sensor chip shown in FIG. 8 is merely illustrative, and is not intended to limit the present invention. According to the different embodiments, at least one layer of the diffusion film 60 with gas screening function and/or the adsorption layer 80 is disposed on the chemical reaction layer 10 by titration-drying method; the air-permeable film 50 with water-blocking property is also formed on the top; the anti-reflection film 40 is attached on one side of the test paper 100 that has not been pretreated.

FIG. 9 provides another manufacturing method. Firstly, four test papers 200a, 200b, 200c, and 200d are provided (step 2-1). The test papers 200a, 200b, 200c, and 200d respectively have a plurality of blocks 201a, 201b, 201c, 201d that do not affect each other. Then, a plurality of sensing portions 202a, 202b, 202c, 202d with different compositions are formed on the blocks 201a, 201b, 201c, 201d respectively (step 2-2). The sensing portions 202a, 202b, 202c, 202d respectively include the coloring reaction layer 20 and the chemical reaction layer 10 described above. Subsequently, cut the test papers 200a, 200b, 200c, and 200d to remove the sensing portions 202a, 202b, 202c, 202d respectively, and combine the sensing portions 202a, 202b, 202c, 202d with a base plate 300 (step 2-3) to dispose the sensing portions 202a, 202b, 202c, 202d on the base plate 300 (step 2-4). In step 2-5, repeat step 2-3 and step 2-4 to obtain a colorimetric sensor chip finally (step 2-6). According to different modes of the embodiment, if necessary, at least one layer of the diffusion film 60 with gas screening function and/or the adsorption layer 80 is provided on the chemical reaction layer 10. The manufacturing method of the colorimetric sensor chip shown in FIG. 9 is merely illustrative, and is not intended to limit the present invention. According to the different embodiment, depending on the requirements, the air-permeable film 50 is attached on the air inlet sides 12a, 12b, and the anti-reflection film 40 is attached on the coloring sides 22a, 22b.

Accordingly, when the colorimetric sensor chip is used to identify whether a meat to be tested is deteriorated, the meat to be tested and the colorimetric sensor chip are placed in a closed environment simultaneously for a period of time, and an odor (such as ammonia) emitted by the meat to be tested enters through the air inlet sides 12a, 12b of the chemical reaction layer 10 and reacts with the reaction zones 13a, 13b to produce a chemical change. Subsequently, the reaction sides 23a, 23b of the coloring reaction layer 20 contact the reaction zones 13a, 13b of the chemical reaction layer 10, so that the coloring indicator contained in the coloring reaction layer 20 shows a specific color corresponding to the chemical change. Therefore, users judge the quality of the meat to be tested through the coloring sides 22a, 22b by their naked eye or machine. The meat to be tested has deteriorated if the color of the meat to be tested is the same as the color shown by the deteriorated meat in a previous database. Alternatively, users can further perform color correction and compare with a calibration curve so that users obtain an ammonia concentration to judge the quality of the meat to be tested by conversion.

What is claimed is:

1. A colorimetric sensor chip comprising a chemical reaction layer and a coloring reaction layer, wherein:
the chemical reaction layer includes at least one reaction zone reacting with a gas to be tested to produce a chemical change, and one side of the chemical reaction layer opposite to the coloring reaction layer is an air inlet side; and
the coloring reaction layer includes a coloring side and a reaction side opposite to each other, the reaction side contacts with the reaction zone of the chemical reaction layer; the coloring reaction layer further includes a coloring indicator to produce a coloring reaction corresponding to the chemical change of the reaction side.

2. The colorimetric sensor chip as claimed in claim 1, wherein the colorimetric sensor chip further comprises a plurality of partition portions, the chemical reaction layer is divided into a plurality of first areas by the plurality of partition portions, the coloring reaction layer is divided into a plurality of second areas by the plurality of partition portions, and the plurality of second areas and the plurality of first areas is stacked corresponding to each other.

3. The colorimetric sensor chip as claimed in claim 1, wherein an anti-reflection film is disposed on the coloring side.

4. The colorimetric sensor chip as claimed in claim 1, wherein an air-permeable film with water-blocking property is disposed on the air inlet side.

5. The colorimetric sensor chip as claimed in claim 4, wherein at least one diffusion film with gas screening function is sandwiched between the air-permeable film and the chemical reaction layer.

6. The colorimetric sensor chip as claimed in claim 5, wherein the diffusion film includes an adsorption molecule.

7. The colorimetric sensor chip as claimed in claim 5, wherein the diffusion film further includes graphenes.

8. The colorimetric sensor chip as claimed in claim 5, wherein a pair of the diffusion films is sandwiched between the air-permeable film and the chemical reaction layer, and an adsorption layer is sandwiched between the pair of the diffusion films.

9. The colorimetric sensor chip as claimed in claim 1, wherein at least one diffusion film with gas screening function is formed on the air inlet side.

10. The colorimetric sensor chip as claimed in claim 1, wherein at least one film layer is further disposed on the air inlet side, the film layer is selected from a group consisting of an air-permeable film with water-blocking property, an adsorption layer, a diffusion film with gas screening function, and combinations thereof.

11. The colorimetric sensor chip as claimed in claim 1, wherein the coloring side is disposed with a colorimetric block.

12. The colorimetric sensor chip as claimed in claim 1, wherein the chemical change is a redox reaction, an acid-base reaction, an enzyme-catalytic reaction, a metal-catalytic reaction, a condensation reaction, a hydrolysis reaction, an addition reaction, an elimination reaction, a substitution reaction, or combinations thereof.

13. The colorimetric sensor chip as claimed in claim 1, wherein the coloring indicator is an acid-base indicator, a solvatochromism, or combinations thereof.

14. The colorimetric sensor chip as claimed in claim 1, wherein compositions of the coloring indicator are selected from a group consisting of a hydrate, a precipitate, a metal complex, and combinations thereof.

15. The colorimetric sensor chip as claimed in claim 1, wherein the chemical reaction layer and the coloring reaction layer form a single-layer structure.
